(19) United States
(12) Patent Application Publication
(10) Pub. No.: US 2015/0196242 A1
(43) Pub. Date: Jul. 16, 2015

(54) Performance Assessment Tool

(71) Applicant: ANTHROTRONIX, INC., Silver Spring, MD (US)

(72) Inventors: Corinna E. Lathan, Wheaton, MD (US); Jack M. Vice, Orlando, FL (US); Joseph Bleiberg, Bethesda, MD (US); James F. Drane, Washington, DC (US); Jonathan S. Farris, Silver Spring, MD (US); Charlotte Jean Smith Safos, Alexandria, VA (US); James Spira, Honolulu, HI (US)

(73) Assignee: ANTHROTRONIX, INC., Silver Spring, MD (US)

(21) Appl. No.: 14/595,667

(22) Filed: Jan. 13, 2015

Related U.S. Application Data
(60) Provisional application No. 61/926,678, filed on Jan. 13, 2014.

(51) Int. Cl. A61B 5/16; G09B 5/00

(52) U.S. Cl. CPC A61B 5/162; G09B 5/00

(57) ABSTRACT

Embodiments of the invention relate to cognitive performance and/or psychological assessment of a subject. Measurement of behavioral status and cognitive efficiency is based upon batteries of tests that include a combination of cognitive and/or psychological tests. A module is provided with a processing unit in communication with memory to administer the cognitive and/or psychological tests and to compute an assessment. Results of the assessment are conveyed on a visual display of the module. In some cases, additional sensor data may be added to the assessment.

### CROSS REFERENCE TO RELATED APPLICATION(S)

This application is a non-provisional patent application claiming the benefit of the filing date of U.S. Provisional Patent Application Ser. No. 61/926,678 filed Jan. 13, 2014 and titled “Performance Assessment Tool” which is hereby incorporated by reference.

### BACKGROUND

The present invention relates to assessment of behavioral functioning. More specifically, the invention relates to a method and system for detecting cognitive and psychological functioning, and any associated limitations.

Methodologies have been developed to determine changes in cognitive efficiency of individuals, including the ability to think and reason. This includes attention, memory and retrieval of information, verbal and spatial processing, speed of processing, reasoning, and judgment. Cognitive assessment is the process of systematically testing a person, analyzing test scores and related data in order to assist healthcare professionals in making judgments about an individual's ability to perform various mental activities involved in the processing, acquisition, retention, conceptualization, and organization of sensory, perceptual, verbal, spatial, and psychomotor information.

Psychological assessment is the process of assessing the extent of impairment to a particular domain of functioning which may have been subject to cognitive impairment due to injury, illness, and/or functional disturbance such as it found in mental illness, sleep impairment, worry, brain injury, neurologic disease, etc. Traditionally, such assessments are conducted by a neuropsychologist trained to evaluate brain function by testing memory, concentration and other abilities, such as language, attention, and spatial skills.

### BRIEF SUMMARY

This invention comprises a method, system, and article for performance degradation and health assessment(s).

In one aspect, an apparatus is provided for performance degradation and health assessment. The apparatus includes a processor in communication with memory and a visual display. A testing module is provided in communication with the memory. The testing module includes a test battery, and more specifically, the testing module supports a simple reaction test, a cognitive test, and a second simple reaction time test. A sensor is provided in communication with the testing module. Data measured by the sensor may be communicated to the processor. Both the simple reaction time test and the choice reaction time test create output data and indicate a basis for performance impairment. Output data from the simple reaction time test and from the choice reaction time tests is stored in the memory, and are independently accessible from the memory.

Other features and advantages of this invention will become apparent from the following detailed description of the presently preferred embodiment of the invention, taken in conjunction with the accompanying drawings.

### BRIEF DESCRIPTION OF THE SEVERAL VIEWS OF THE DRAWINGS

The drawings referenced herein form a part of the specification. Features shown in the drawings are meant as illustrative of only some embodiments of the invention, and not of all embodiments of the invention unless otherwise explicitly indicated.

FIG. 1 depicts a block diagram of a testing module.

FIG. 2 depicts a screen shot for a Simple Reaction Time Test, showing a stimulus on a visual display of the testing module.

FIGS. 3A and 3B depict screen shots for a Procedural Reaction Time Test, showing a visual display with indicia.

FIG. 4 depicts a screen shot of a Spatial Processing Test.

FIGS. 5A and 5B depict a screen shot of a Code Substitution Test.

FIG. 6 depicts a screen shot of a Go-NoGo Test.

FIG. 7 depicts a flow chart illustrating a process for calculating a composite score the test battery.

FIG. 8 depicts a flow chart illustrating a process for measuring a testing module for latency.

FIG. 9 depicts a block diagram illustrating a sample result scale.

FIG. 10 depicts a block diagram of a sample Full Report.

FIG. 11 depicts a block diagram of the assessment kit.

FIG. 12 depicts a block diagram illustrating tools embedded in a testing module to support administration of neuro-cognitive and/or psychological assessment.

FIG. 13 is a block diagram of an example match to sample test in a visual display.

FIG. 14 is a block diagram of an example memory search test in a visual display.

FIG. 15 is a flow chart illustrating one aspect of assessing cognitive efficiency.

FIG. 16 is a flow chart illustrating comparison of the simple reaction times based on the sequential administration of tests shown in FIG. 15.

FIG. 17 is a flow chart illustrating a cognitive metering device employed with assessment data.

FIG. 18 is a flow chart illustrating a process for calibrating or re-calibrating the metering device.

FIG. 19 is a flow chart illustrating a process for employing the cognitive assessment device with one or more embedded hardware sensors.

FIG. 20 is a flow chart illustrating a process for interacting with the portable assessment device, with the interaction affecting the state of operation of the device.

### DETAILED DESCRIPTION

It will be readily understood that the components, as generally described and illustrated in the Figures herein, may be arranged and designed in a wide variety of configurations. Thus, the following detailed description of the embodiments of the apparatus, system, and method, as presented in the Figures, is not intended to limit the scope of the invention, as claimed, but is merely representative of selected embodiments.

The functional unit described in this specification with elements labeled as managers. A manager may be implemented in programmable hardware devices such as field programmable gate arrays, programmable array logic, programmable logic devices, or the like. The manager may also be implemented in software for execution by various types of processors. An identified manager of executable code may, for instance, comprise one or more physical or logical blocks of computer instructions which may, for instance, be organized as an object, procedure, function, or other construct. Nevertheless, the executables of an identified manager need not be physically located together, but may comprise disparate instructions stored in different locations which, when joined logically together, comprise the manager and achieve the stated purpose of the manager.

Indeed, a manager of executable code could be a single instruction, or many instructions, and may even be distributed over several different code segments, among different applications, and across several memory devices. Similarly, operational data may be identified and illustrated herein within the manager, and may be embodied in any suitable form and organized within any suitable type of data structure. The operational data may be collected as a single data set, or may be distributed over different locations including over different storage devices, and may exist, at least partially, as electronic signals on a system or network.

Reference throughout this specification to “a select embodiment,” “one embodiment,” or “an embodiment” means that a particular feature, structure, or characteristic described in connection with the embodiment is included in at least one embodiment. Thus, appearances of the phrases “a select embodiment,” “in one embodiment,” or “in an embodiment” in various places throughout this specification are not necessarily referring to the same embodiment.

Furthermore, the described features, structures, or characteristics may be combined in any suitable manner in one or more embodiments. In the following description, numerous specific details are provided, such as examples of recovery manager, authentication module, etc., to provide a thorough understanding of embodiments. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

The illustrated embodiments will be best understood by reference to the drawings, wherein like parts are designated by like numerals throughout. The following description is intended only by way of example, and simply illustrates certain selected embodiments of devices, systems, and processes that are consistent with the invention as claimed herein.

Behavioral test methodologies have been developed to determine changes in psychological and cognitive functioning of individuals. A behavioral assessment is a process of systematically testing a person, analyzing test scores and related data in order to assist healthcare professionals making judgments about an individual's diagnosis, treatment, and level of function in daily living. The behavioral assessment can also include measurements of problem-solving abilities (or cognitive efficiency) such as speed and accuracy. Through the cognitive tests, the tool assesses neuro-cognitive function as related to neurologic injury, neurologic disease, and other stressors. Through the psychological tests, the tool assesses symptoms of depression, post-traumatic stress, insomnia, anger, and post-concussive syndrome.

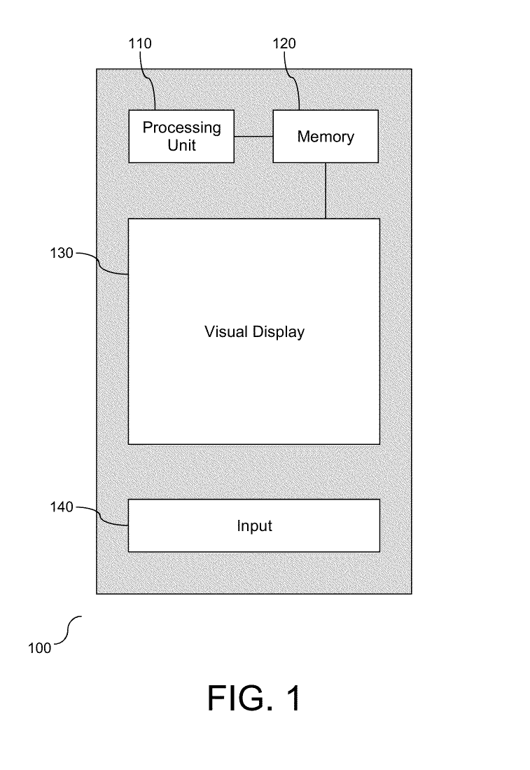

FIG. 1 is a block diagram of a testing module (100). As shown, the module (100) is provided with a processing unit (110) in communication with memory (120) and a visual display (130). More specifically, the module presents an assessment in the form of a combination of neuro-psychological and neuro-cognitive tests on the visual display (130). The user responds to the assessment through an input device (140). More specifically, at least one cognitive test battery is made available to a user through the testing module (100). The user responds to those tests using the input device (140) as described in detail below.

The module (100) shown in FIG. 1 is a portable module that may provide assessment at any location. The assessment is based on a combination of tests that assess various cognitive and/or behavioral impairments, such as but not limited to cognitive functioning, sleep, mood, posttraumatic stress, daily functioning, as well as level of motivational effort. The behavioral tests include a battery of one or more tests provided to a subject to assess if there is a psychological impairment and the cause thereof. Similarly, the neuro-cognitive tests include a battery of tests provided to a subject to assess a cause of cognitive impairment. From a library of potential tests on the device, several test batteries can be configured. One test battery can included several neuro-cognitive tests to be used for a brief screening following a concussion. Another test battery can include both several neuro-cognitive tests and psychological screening tools be used as a brief screening to help identify suspected impairment, including but not limited to concussion, depression or post-traumatic stress disorder, and exhaustion. Still another battery comprised of up to a dozen neuro-cognitive and behavioral tests to assist healthcare professionals to determine the specific cause and level of a person's impairment.

Many such batteries from the library of tests can be configured in order to accommodate the needs of the healthcare professional. A clinician or trained personnel may employ a configured module to provide screening of the subject in the environment in which they operate or received an injury, or else in a specialized medical clinic. The output from the assessments and their associated batteries of tests can provide an output with an indicator to assist the healthcare professional in their initial assessment of the subject's level of functioning in a variety of neuro-cognitive and/or psychological domains. For example, in one configuration, the output may include indicia in the form of a color coded chart, with green indicating the subject is in a normal range, yellow indicating there is a possibility of an impairment that may need further analysis, and red suggesting the possibility of impairment that may require a further assessment and possibly treatment of the tested person.

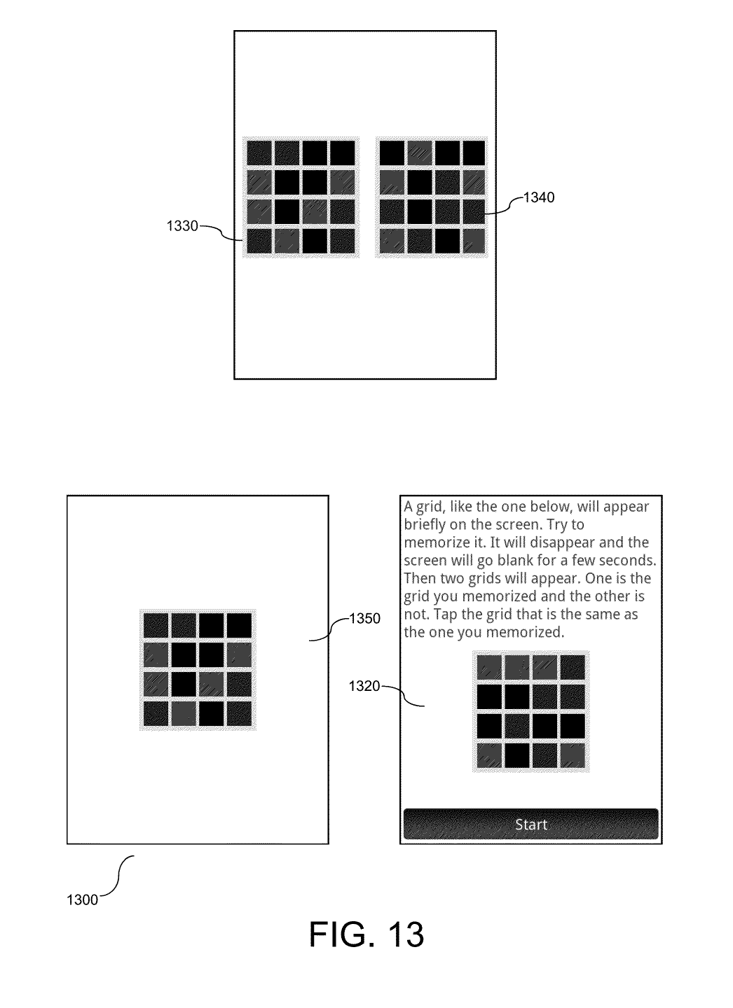

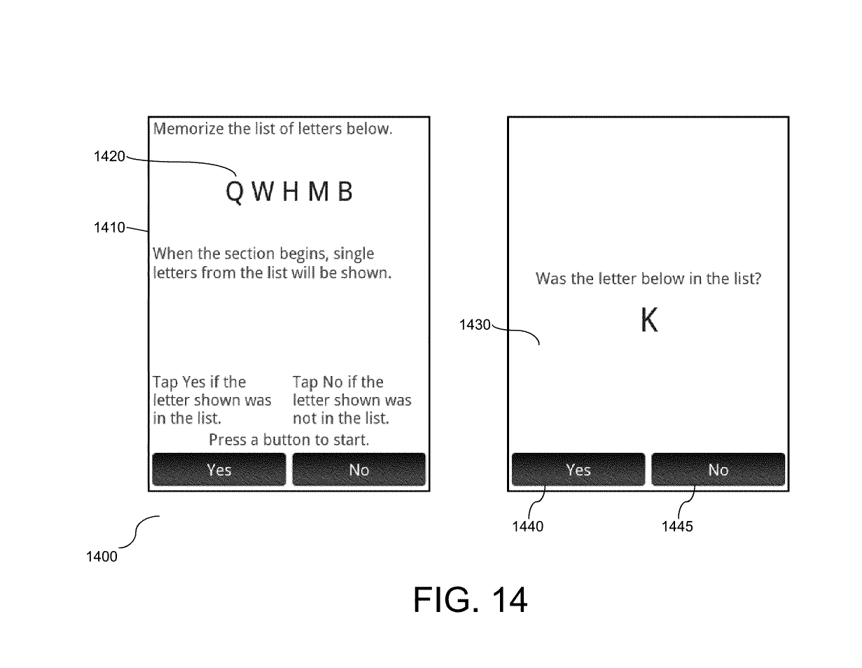

Cognitive assessment includes one or more of the following tests: Simple Reaction Time, Procedural Reaction Time, Spatial Processing, Code Substitution, Go-NoGo, Memory Search, and Match to Sample. FIG. 13 is a block diagram (1300) of an example match to sample test in a visual display. Specifically, a first visual display (1310) provides a first grid (1320) with a plurality of boxes and a pattern therein. After a short period of time, the visual display will be blank and then the visual display will exhibit two grids (1330) and (1340). One of the two grid exhibited will be the original grid and the second grid will have a different pattern. The subject of the test will be required to select one of the grids—the grid with the matching pattern as the first grid (1320) is the correct answer (1350). FIG. 14 is a block diagram (1400) of an example memory search test in a visual display. A visually display (1410) exhibits a list of alphanumeric characters (1420). In a second section of the visual display (1430), a field is provided to display one alphanumeric character. Two input fields are provided (1440) and (1445), one to indicate the displayed alphanumeric character matches one of the letters that was presented in the list and another to indicate the displayed alphanumeric character does not match of the letter that was presented in the list. The behavioral assessment includes one or more of the following tests: PHQ-9, PC-PTSD, ISI, PSQI, and PCL-M. In one embodiment, additional tests may add to the selection of both the cognitive and psychological assessment from which various configurations can be made into a battery. For example, different batteries of tests may be configured from a library of tests.

The following is a description of the cognitive tests:

### Cognitive Tests:

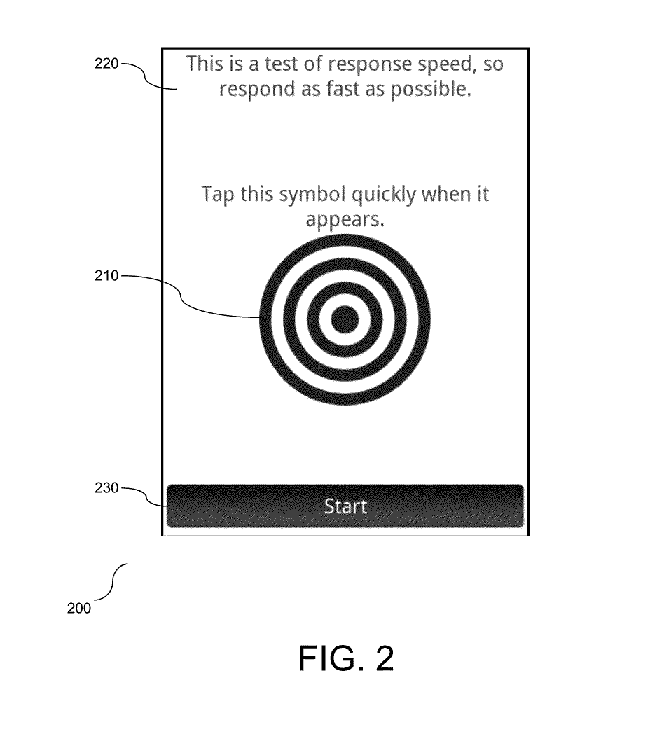

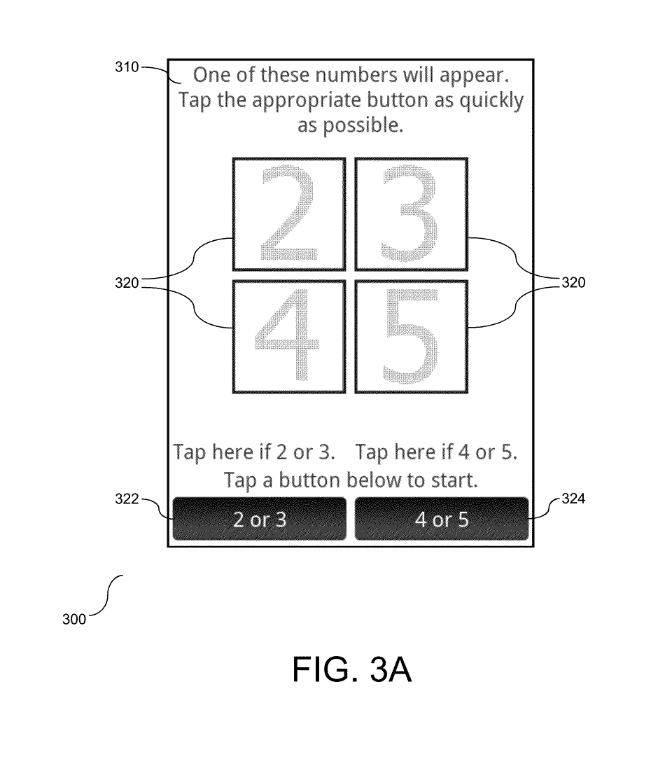

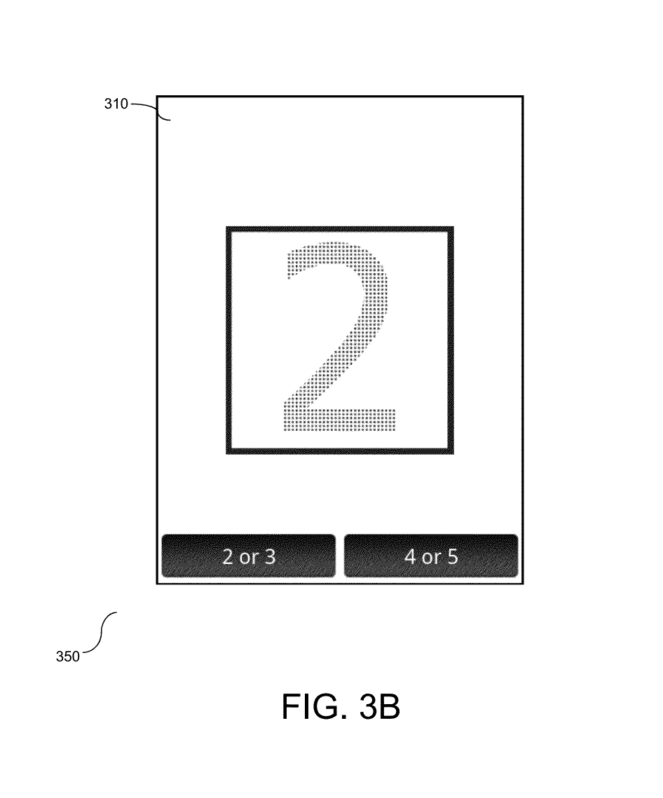

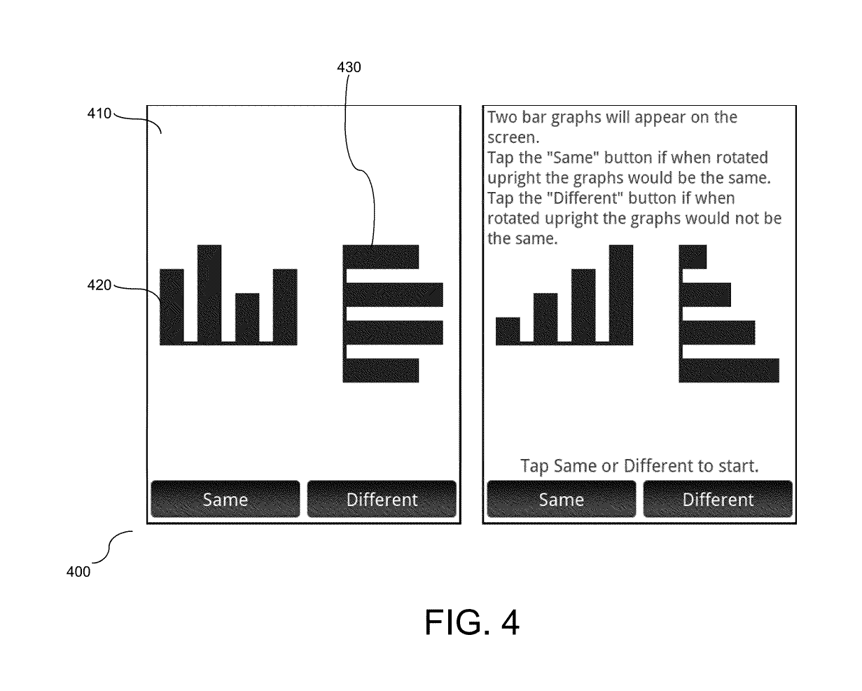

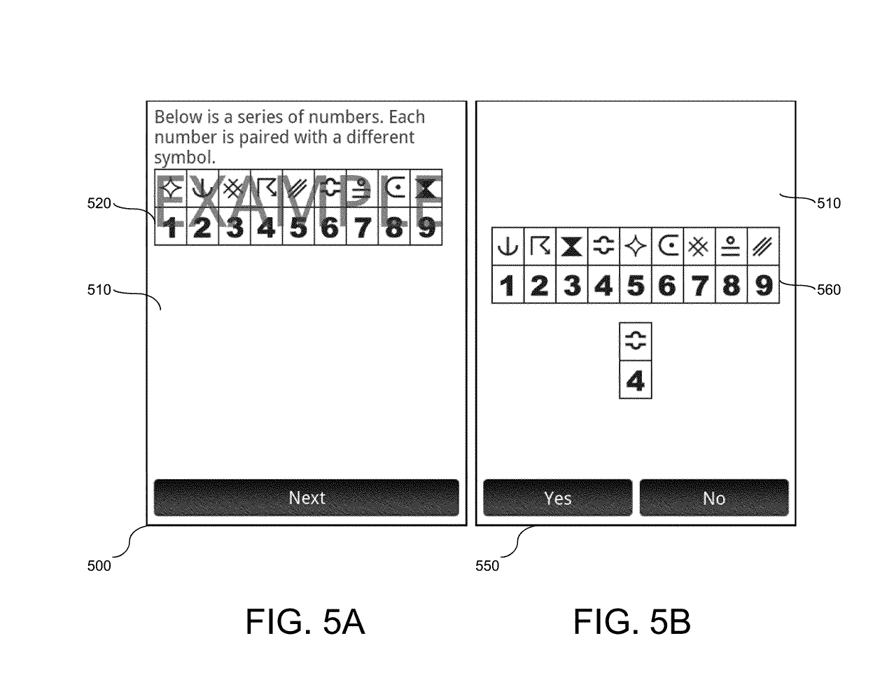

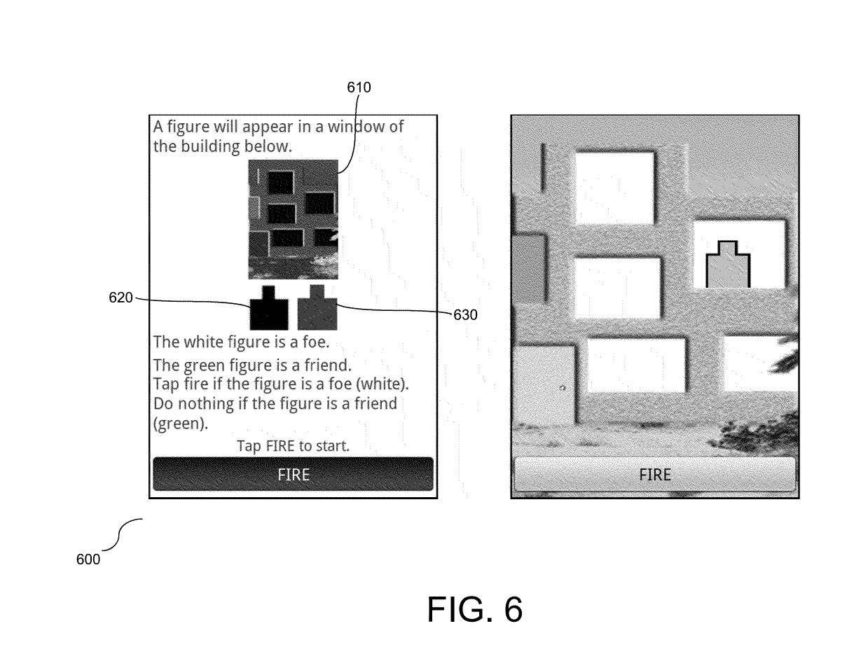

1. Simple Reaction Time: This test requires a participant to react to a visual stimulus on a visual display. More specifically, when the stimulus is present, the participant employs an input device. The input device may include an implement, to tap on the stimulus. Similarly, in an embodiment where the visual display is a touch screen, the input device may be a finger of the participant. The tests measures the time from when the stimulus is presented on the visual display until the time the input device touches the stimulus on the visual display. In one embodiment, the simple reaction time test is an assessment of psycho-motor speed. FIG. 2 is a screen shot (200) of a stimulus (210) on a visual display (220). The stimulus is shown in this example as a bulls-eye, but is not limited to this physical form. In one embodiment, the stimulus will appear a set number of times, requiring the participant to respond to each presentation, with time measured for each interval from presentation to response. In one embodiment, in input button (230) is provided on the visual display (220).
2. Procedural Reaction Time: This test requires a participant to differentiate between two character sets. More specifically, a stimulus is presented to the participant, and the participant employs an input device to input a selection to convey their reaction to the stimulus. FIG. 3 is a screen shot (300) of a visual display (310) with indicia (320) indicative of instructions. In the example shown herein, the instructions convey that one of four numbers will appears on the visual display (310). Two inputs are provided (322) and (324), with input (322) to be activated in response to a first set of indicia, e.g. if the number on the display is a 2 or a 3, and input (324) to be activated in response to a second set of indicia, e.g. if the number on the display is a 4 or a 5. FIG. 3B is a screen shot (350) of a visual display (310) with the test showing indicia in the form of a number 3. Test measurements include both selection of the choice, and time to enter the selection, and may be referred to herein as a choice reaction time test. Accordingly, accuracy of choice and an associated time interval are assessed in the procedural reaction time test.
3. Spatial Processing: This test requires a participant to differentiate between two visual presentations. More specifically, at least two graphical elements are presented to the participant, and the participant indicates if the two elements are the same. FIG. 4 is a screen shot (400) of a visual display (410) with two graphical elements (420) and (430). In the example shown herein, the graphical elements are histograms. However, the invention should not be limited to this implementation. In another embodiment, the graphical elements may be other formations. The first and second graphical elements (420) and (430) are identical with respect to size and shape. The first graphical element (420) has a first alignment, and the second graphical element (430) is rotated 90 degrees in a clockwise direction. The test is for the participant to determine of the two graphical elements are equivalent. Accordingly, the results of the test are indicative of any impairment with respect to spatial processing.
4. Code Substitution Learning: This test requires a participant to memorize a pattern and to recall the pattern during an assessment. FIG. 5A is a screen shot (500) of a visual display (510) prior to assessment, the visual display showing a graphical element (520). In the example shown herein, the graphical element (520) includes a set of symbols and digits, with each symbol paired with a digit. The graphical element (520) functions as a key for the assessment. FIG. 5B is a screen shot (550) of the visual display (510) during the assessment. A graphical element (560) in the form of a single digit is paired with a single symbol. The participant indicates whether or not the paired digit and symbol (560) matches a pair that was presented in the graphical element (520) on the visual display (510). Accordingly, the test assesses the ability to match a simple pattern with a key.
5. Code Substitution Recall: This test requires a participant to memorize a pattern and to recall the pattern during an assessment. More specifically, the simple pattern is presented to the participant without the key. The participant indicates whether or not the pattern was presented in the learning phase of the test, e.g. as shown FIG. 5A. Results may vary based upon diligence and effort.
6. Go-NoGo: This is a reaction time, forced choice tasks. FIG. 6 is a screen shot (600) from the visual display. As shown, a building (610) is presented on a visual display (640), with the building showing a plurality of windows. One of two icons may appear in any one of the windows in the building (610). In one embodiment, one icon representing a friend (620) and a second icon representing a foe (630). The participant must activate a button in communication with the visual display (640) when the second icon (630) appears in one of the windows. The tests assess both speed and accuracy, and can suggest impulsivity. The test has sufficient trials to determine speed and accuracy of targets, omissions, and commissions in order to derive a de-sensitivity metric, as found in continuous tasks.
7. Memory Search: This test assesses executive functioning and short-term memory.

The subject memorizes a set of five letters, after which letters on the screen appear one at a time. The subject determines if the letter on the screen is a member of the memory set of five letters.
8. Match to Sample: This test measures short-term memory, attention, and visual spatial discrimination. A single 4×4 checkerboard pattern is presented on the screen for brief study period. It then disappears for five seconds, after which two patterns are presented side-by-side. The subject indicates which of these two patterns matches the first checkerboard pattern.

The following is a description of select psychological assessments:
1. Deployment Stress Inventory (DSI): This test presents a series of statements that include PTSD and chronic pain as well. The participant is asked, “How often do you have this problem?” with the responses almost never, sometimes, and often or constantly.
   The list of tests may be expanded to include additional questions or shortened due to removal or elimination of an item from this test. The following are a list of possible questions in the DSI test:
      How often do you have these problems?
      Headaches
      Body pain other than headache
      Pain interfering with work
      Blurred or double vision
      Changed taste: dulled or absent
      Poor balance or coordination
      Changed Smell: dulled or absent
      Dizziness or vertigo (room spinning)
      Sick to your stomach or vomiting
      Problems falling asleep
      Problems staying asleep
      Difficulty staying alert at work
      Disturbing dreams or nightmares
      Thinking you would be better off dead
      Tiredness “more than usual”
      Slowed thinking or performing
      Quick to anger, angry outbursts
      Difficulty completing routine work
      Difficulty remembering things
      Feeling nervous, anxious, or jittery
      Jumpy, easily startled
      Feeling nobody cares about you
      No emotions or just feeling numb
      Feeling sad or discouraged
      Unwanted thoughts or flashbacks
      Loosing focus or concentration
      Thoughts of hurting yourself
2. Psychological Health Questionnaire (PHQ-9): The Patient Health Questionnaire (PHQ) is a self-administered version of the PRIME-MD diagnostic instrument for common mental disorders. The PHQ-9 is the depression module, which scores each of the 9 DSM-IV criteria on a scale with “0” (not at all) to “3” (nearly every day).
3. Primary Care PTSD (PC PTSD): The PC-PTSD is a 4-item screen that was designed for use in primary care and other medical settings. The screen includes an introductory sentence to cue participants to traumatic events. In most circumstances the results of the PC-PTSD should be considered “positive” if a participants answers positively to any three items. Those screening positive should then be assessed with a structured interview for PTSD. The screen does not include a list of potentially traumatic events.
4. Pittsburgh Sleep Quality Inventory (PSQI): The PSQI is composed of nineteen self-rated questions and five questions rated by a bed partner or roommate (only the self-rated items are used in scoring the scale). The self-administered scale contains fifteen multiple-choice items that inquire about frequency of sleep disturbances and subjective sleep quality and four write-in items that inquire about typical bedtime, wake-up time, sleep latency, and sleep duration. The five bed partner questions are multiple-choice ratings of sleep disturbance. All items are brief and easy for most adolescents and adults to understand. The items have also been adapted so that they can be administered by a clinician or research assistant.
5. Post-Traumatic Stress Disorder Check List—military version (PCL-M): The use of the Post-Traumatic Stress Disorder Checklist (military version—PCL-m) is recommended for PTSD screening. The PCL-m is comprised of seventeen items matching categories B, C, and D of the DSM-IV criteria for PTSD. It was developed by the National Center for PTSD for use in civilians (PCL-c) and military members (PCL-m). In one embodiment, measures are part of the library from which batteries have been assembled. Criteria based on research with specific populations are used to suggest degree of impairment (some being more sensitive for screening purposes, and some being more specific for diagnostic support).
6. Insomnia Severity Index: The Insomnia Severity Index is reliable and valid and has seven items that use a five-point Likert-style scale. Scores can range from 0 to 28, with a cutoff score of 8 suggesting the presence of clinical insomnia. The questionnaire has three questions assessing the severity of insomnia and one question each assessing satisfaction with current sleep pattern, sleep interference, “noticeability” of sleeping problem to others, and concern about sleeping problems.

In one embodiment, the assessment is not individualized. More specifically, a selection of questions pertaining to each of the specified categories may be mixed together. An advantage of combining different categories of questions into a single assessment provides a combined picture of different categories of potential concerns pertaining to the subject participant.

As shown above, there are various cognitive and psychological tests. Different combinations of tests may be administered depending upon the scenarios. The following description(s) pertain to examples of such scenarios. A first line of care includes a first battery of tests, also referred to herein as rapid tests. The following tests are administered in the first battery: Simple Reaction Time, and Choice Reaction Time Tests. The tests in this first battery are cognitive efficiency reaction time tests. The first line of care is intended to be administered in the field proximal to the time of injury (typically within 24 hours of suspected concussion), and includes both of the described tests. Results of the test (as described below) are indicative of the immediate care required, e.g. supports the healthcare provided in assessing if a further assessment or treatment may be required.

A second line of care includes a second battery of test in the form of a combination of cognitive and psychological tests, also referred to herein as brief tests. The following tests are administered in the second battery: Simple Reaction Time, Procedural Reaction Time, Spatial Processing, Code Substitution, Go-NoGo, PHQ-9, PC-PTSD, and ISI. The second line of care can be administered at least 24 hours following after a suspected concussion, or at any time due to any suspected impairment of functioning, such as disturbed mood, exhaustion, pain, etc. The first and second line batteries described above are intended for screening purposes in order to suggest the need for further evaluation by a specialized healthcare professional. These first two test batteries can be utilized by provider-extenders (medics, corpsman, psych techs, medical assistants, nurses, etc.) under the guidance of a licensed healthcare professional.

A third line of care includes a third battery of tests, including a more in depth combination of cognitive and behavioral tests, also referred to herein as standard tests. The following tests are administered in the third battery: Simple Reaction Time, Procedural Reaction Time, Spatial Processing, Code Substitution, Go-NoGo, Memory Search, Match to Sample, PHQ-9, DSI, PSQI, and PCL-M. The third battery of tests is intended to be administered at least forty-eight hours or more after a suspected concussion or at any time due to suspected impairment from any cause (lingering effects from an earlier concussion, mood disturbance such as posttraumatic distress or depression, or exhaustion due to cumulative stress or insomnia). This battery includes each of the described tests. Whereas the first two batteries can be delivered in any environment, such as where the injury occurred by a provider-extender, this third battery is intended to be delivered in a traditional healthcare setting by a more senior healthcare professional, typically a licensed healthcare provider. It is intended to assist the healthcare professional to more specifically determine the extent of impairment and the specific causes of the impairment so that a diagnosis and recommendation for treatment can be more accurately made by that healthcare professional. Other configurations are available as well, including a Clinic Version that includes several functional tests, and can select Neuro-Cognitive tests only, Psychological tests only or each test separately, as needed by the healthcare provider. For example, in one embodiment, the participant cannot select among the tests to be administered in each battery, and must attend to each of the tests therein.

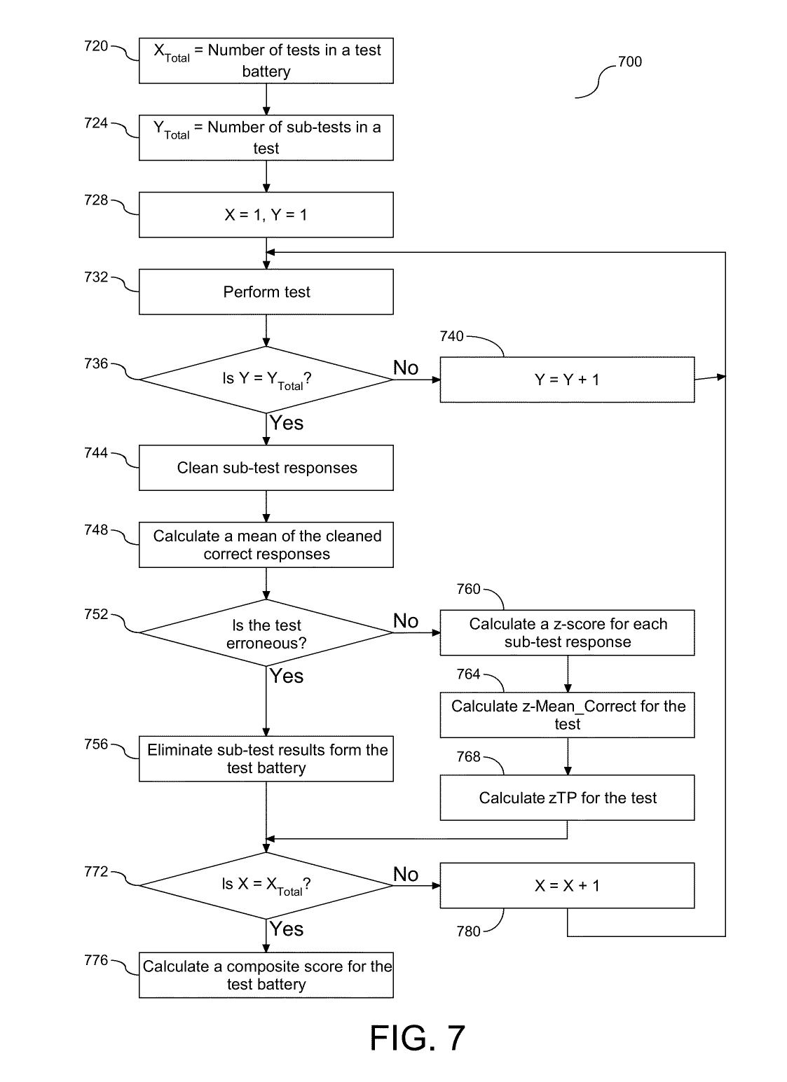

Each battery of tests may generate a composite score by calculating an average of the normalized throughput scores for each test in the test battery. FIG. 7 is a flow chart (700) illustrating a process for calculating a composite score for the test battery. In one embodiment, a test battery includes a plurality of tests, which include a plurality of individual subtests yielding test responses. To begin testing, the total number of tests to be performed in a test battery is identified (720), and the total number of subtests to be administered to yield test responses is identified (724). The first subtest for the first test in the test battery is initialized (728). After a subtest is administered (732), it is determined if all of the subtests have been administered (736). A negative response to the determination at step (736) is followed by an increment of the counting variable (740) and a return to step (732). However, a positive response to the determination at step (736) is followed by cleaning the subtest responses (744).

Each subtest response may be “cleaned” to eliminate erroneous responses from the performed test. In one embodiment, erroneous responses may include wrong responses. In another embodiment, erroneous responses may include anomalies such as too fast responses, e.g. faster than 150 milliseconds or, for a single reaction time test, too slow responses, e.g. slower than 650 milliseconds. Following step (744), a mean and median of the resulting cleaned correct responses is calculated (748). In one embodiment, a median correct reaction time is calculated. Following step (748), each test is evaluated to determine if the test is erroneous (752). In one embodiment, erroneous tests are those tests administered with more than thirty percent of trials missing. In another embodiment, erroneous tests are those tests with an average percent correct less than sixty-six percent, as the responses may be approaching chance. A positive response to the determination at step (752) is followed by eliminating the subtest responses and the test from the test battery (756). Following step (756), it is determined if all of the tests have been administered for the test battery (772). A negative response to the determination at step (772) is followed by an increment of the counting variable (780) and a return to step (732). However, a positive response to the determination at step (772), which indicates a completion of all of the tests in the battery, is followed by calculating the composite score for the test battery (776), as described below. A negative response to the determination at step (752) is followed by calculating additional metrics for the test.

A mean correct score and percent correct will be calculated for each test for use in calculating the throughput. A z-score is calculated for each subtest response using the test mean (760). A z-Mean_Correct is derived from the z-score as the mean of the z-scores for the subtest responses (764). A z-score for the throughput (“zTP”) is calculated for each test (768). In one embodiment, the zTP is calculated by dividing the percent correct for a test by the z-Mean_Correct for a test and multiplying the resulting quotient by 60,000. This calculation yields a standardized score for each test in a test battery equivalent to the number of correct answers in a minute.

Following step (768), it is determined if all of the tests have been administered for the test battery (772). A negative response to the determination at step (772) is followed by an increment of the counting variable (780) and a return to step (732). However, a positive response to the determination at step (772) is followed by generating the composite score for the test battery (776). In one embodiment the composite score equals the average zTP for the test battery. Specifically, the sum of the zTPs for each test in a test battery is divided by the number of tests in the test battery. To that end, responses from different tests using different standards of measure in a test battery have been normalized to generate a metric for the test battery, as a whole. The composite score may be stored in memory, or in one embodiment may be graphically displayed on an associated visual display.

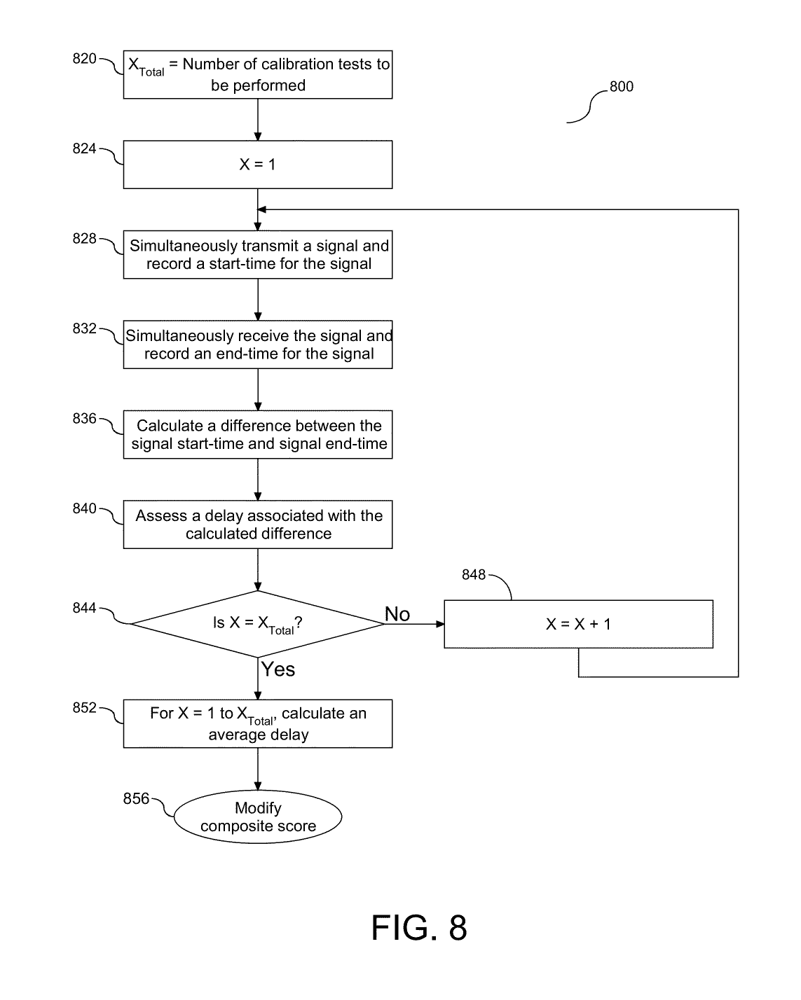

FIG. 8 is a flow chart (800) illustrating a process for measuring a testing module for latency. The latency assessment may be in the form of light or sonic, or a combination thereof, both of which account for time latency. The test utilizes the testing module and a transmitter. The testing module is in communication with the transmitter. In one embodiment, the transmitter is directly related to an interface of the testing module. A sonic transmitter employs a sonic signal, and a light transmitter is a surface with a reflective property, such as a mirror, and employs a light signal. The assessed latency considers the transmission of a signal and any latency associated with the transmission or receipt of the signal by the testing module.

To begin testing, the total number of calibration tests to be performed in a test sample is identified (820), and the first test in the test sample is initialized (824). The transmitter transmits the signal, and a start-time for the transmitted signal is recorded. (828). In one embodiment, the start-time is recorded by a time management application. In one embodiment, the recording of the start-time takes place simultaneous or near simultaneous with the start of the signal. The transmission may be a sonic signal, that is, relating to sound waves, such as a sound at a set or variable frequency, or a light signal, such as a reflection of the testing module in a transmitter surface. The testing module may decide whether the testing module is testing for a sonic signal delay or a light signal delay. The signal is received by a receiver, and an end-time for the received signal is recorded (832). In one embodiment, the end-time is recorded by a time management application. In one embodiment, the recording of the end-time takes place simultaneous or near simultaneous with the receipt of the signal. In one embodiment, the receiver is a sonic signal receiver application, such as a microphone. In another embodiment, the receiver is a light capture application, such as a camera. In one embodiment, the receiver is an application on the testing module.

A difference between the start-time and end-times of the signal is calculated for the test to assess a signal delay associated with the calculated difference (836). In one embodiment, for a sonic signal, the difference is calculated and the speed of sound is subtracted from the difference. In another embodiment, for a light signal, the difference is calculated and the speed of light is subtracted from the difference. Accordingly, for each test, the start-time and end-times are captured and any associated signal delay is recorded. In one embodiment, the signal delay is the absolute value of the start-time and end-time. To calculate the latency associated with the testing module, the signal delay is adjusted to account for outside influences (840). In one embodiment, the signal delay is adjusted to account for signal noise. For example, one or more signal parameters are adjusted to account for signal noise, such as ambient signal noise, and the adjustment may include a modification of the signal wavelength for a sound or light signal.

The signal delay is employed to assess testing module latency. Following step (840), it is determined if all of the calibration tests have been completed (844). A negative response to the determination at step (844) is followed by an increment of the counting variable (848) and a return to step (828). However, a positive response to the determination at step (844) is followed by calculating an average delay for the test sample (852). In one embodiment, the average delay calculation considers variation in the test results, specifically, the time distribution. Once the average delay for the testing module is calculated, the composite score for the test battery executed on the testing module is modified to reflect any testing module latency (856). Accordingly, the illustrated process may be used to maintain accuracy of the composite score.

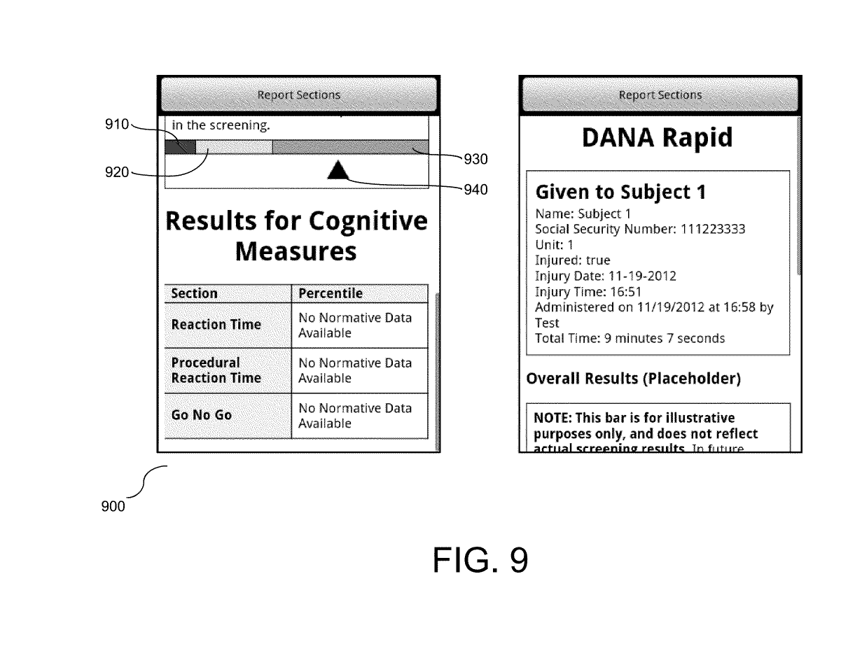

Each of the battery of tests produces a report to convey output from the battery of tests that was administered to a participant. For each battery there may be three compilations of data reported, including a basic report, a full report, and a raw data file. The basic report is employed to convey a timely assessment of functioning for the provider to use in determining fitness for continued activity or referral for more in-depth assessment and potential treatment. In one embodiment, the basis report includes a color coded scale with marker to indicate the assessment results in the scale. FIG. 9 is a block diagram (900) illustrating a sample result scale. As shown, the scale is a form of a bar graph with three sections (910), (920), and (930). In one embodiment, each section is represented by a different color, including a first section (910) represented by the color red, a second section (920) represented by the color yellow, and a third section (930) represented by the color green. A cursor (940) is positioned adjacent to the bar graph indicating a position of the results in the graph. In one embodiment, positioning of the cursor (940) adjacent to any portion of the third section (930) is an indication that the participant does not seem to be impaired in that domain, positioning of the cursor (940) adjacent to any portion of the first section (910) is an indication to the provider that the participant responded in a way that is consistent with impairment, and positioning of the cursor (940) adjacent to any portion of the second section (920) is an indication that the participant's responses are inconclusive, requiring additional observation and possibly retesting at another time. Accordingly, the basic report provides limited feedback and is generally employed for a quick assessment of the participant.

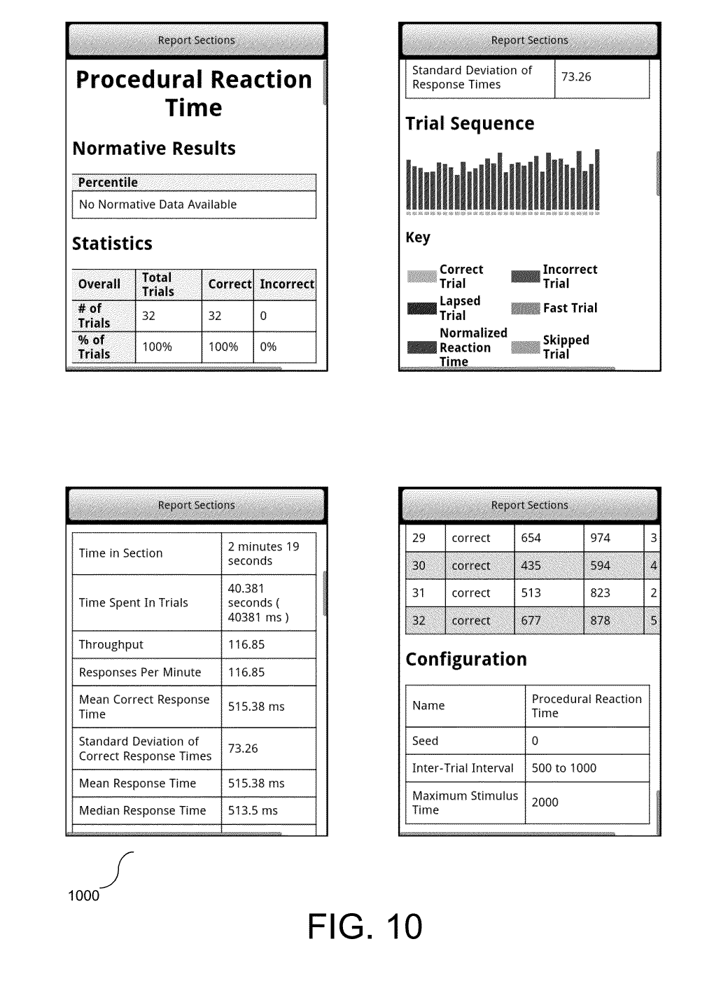

A second report generated by the module is known as a Full Report, and it provides information for a health service provider at various levels of detail so that the provider can drill down to the level that is most beneficial to them. This includes general and separate summaries of cognitive and behavioral measures (useful for most clinicians) all the way down to individual responses (useful for specialists and researchers). In one embodiment, the provider can access data such as reaction time and accuracy for a cognitive test, or the summary score for the Post Traumatic Stress Disorders Checklist (PCL-M). FIG. 10 is a block diagram (1000) of a sample Full Report. In one embodiment, a third report may generated by the module is known as a Raw Data File, and it provides an exact measurement of all responses for all activities. This is useful for programmers, researchers, and specialists, to insure quality control.

Each of the battery of tests described herein may be applied to different environments to aid in the assessment of injury and or fitness for activity. In one embodiment, the screening battery of tests may be employed in a military operational environment for screening of potential injury assessment of a soldier's functioning in the line of duty. In another embodiment, the screening battery of tests may be employed in a commercial environment, such as athletics and associated injury to athletes. In still other embodiments, the more in-depth battery of tests can be used for the military in a war zone at an Aid Station, or in a traditional clinical setting by advanced healthcare providers.

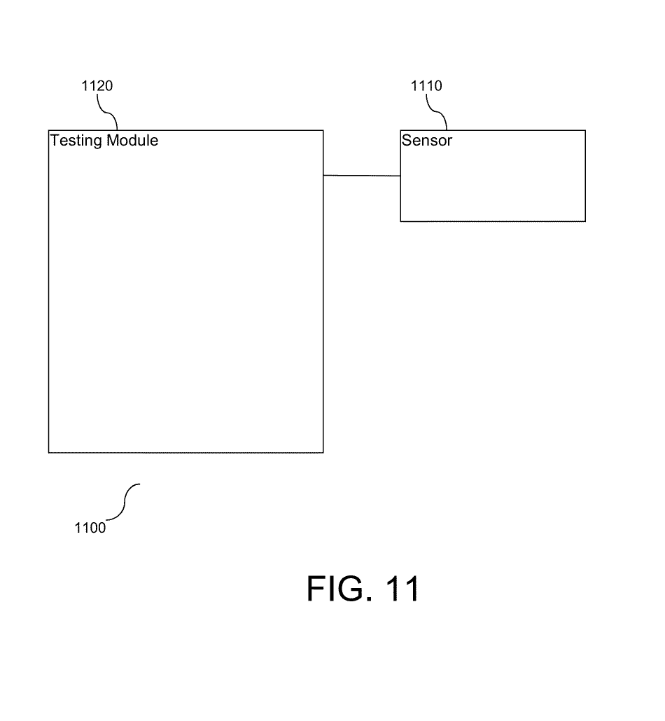

With respect to application of the module in different settings, e.g. commercial or military, an assessment kit may be configured that includes a sensor (1110) in communication with the module (120). In one embodiment, the sensor may also be applied in a military environment. Similarly, in one embodiment, the sensor functions as an input device. FIG. 11 is a block diagram (1100) of the assessment kit. The sensor (1110) is applied to a secondary surface in communication with the participant. A secondary surface, such as a helmet, may be used to measure cranial movement in view of impacts on the helmet by, for example, sound or blast waves from an explosion. In one embodiment, the sensor may be applied to clothing, a helmet, etc., and measure impact and/or acceleration, which may be used to as factor for impairment evaluation. Similarly, in one embodiment, the sensor (1110) is a dual axis sensor to sense data on at least two axes, and in a further embodiment, the sensor (1110) is a tri-axis sensor to sense data on at least three axes. The sensor (1110) may measure one or more of the following forms of data: balance, orientation, impact, biomarkers, and neuronal activity. In one embodiment, e.g. for impact, the sensor (1110) is activated in response to receipt of a physical stimulus that exceeds a threshold, e.g. activation of the sensor. Similarly, in one embodiment, the sensor may be activated in response to receipt of continuous data, such as a balance assessment. Once the sensor is activated, a signal or indicia is conveyed to indicate that testing through use of the testing module (1120) is recommended. In one embodiment, the signal or indicia is different for each axis of the sensor. The sensor signal or indicia includes, but is not limited to, a visual signal, an auditory signal, and/or a communication signal. Details of the testing module (1120) are provided in FIG. 1. Accordingly, a kit is disclosed with inclusion of a sensor to provide indicia to initiate assessment through use of the module.

As indicated above with respect to the kit, the module may be applied in various environments, including military and athletics. With respect to the athletic environment, it may be warranted to assess the participant for initial signs of a concussion or other head related injury. In one embodiment, the module is configured to provide a standardize assessment of concussion (SAC) which measures: Orientation (month, date, day of week, year, time), Immediate Memory, Neurologic Screening, Loss of consciousness, Amnesia, Strength, Sensation, Coordination, Concentration, Exertional Maneuvers, and Delayed Recall. One embodiment includes the military equivalent version of the SAC called the Military Acute Concussion Evaluation (MACE). The module employed herein delivers the SAC and MACE digitally. In addition, one embodiment includes the use of the sensor to quantify the balance score, such as the Balance Error Scoring System (BESS). Specifically, the module measure and automatically calculates using the sensors to measure balance during administration of the BESS to quantify balance. The SAC and the BESS are generally administered as part of the SCAT2-Sport Concussion Assessment Tool 2. The use of the SCAT-2 has value in helping sports medicine professional in the diagnosis and management of conditions in athletes on the sport sideline, particularly in identifying concussions. In one embodiment, the SCAT-2 may also be applied to military personnel in the field. The SCAT-2 is designed for rapid concussion evaluation on the sidelines. The SCAT-2 includes the SAC, a brief neurocognitive test battery that assesses attention and memory function, but the SCAT-2 is not intended to replace comprehensive neurocognitive testing or used as a stand-alone tool for the ongoing management of sports concussions. It is also important to remember that symptoms may not appear until several hours after injury. Accordingly, the SCAT-2 may be a test that is employed as a preliminary assessment, followed by one of the three batteries of tests configured with the module.

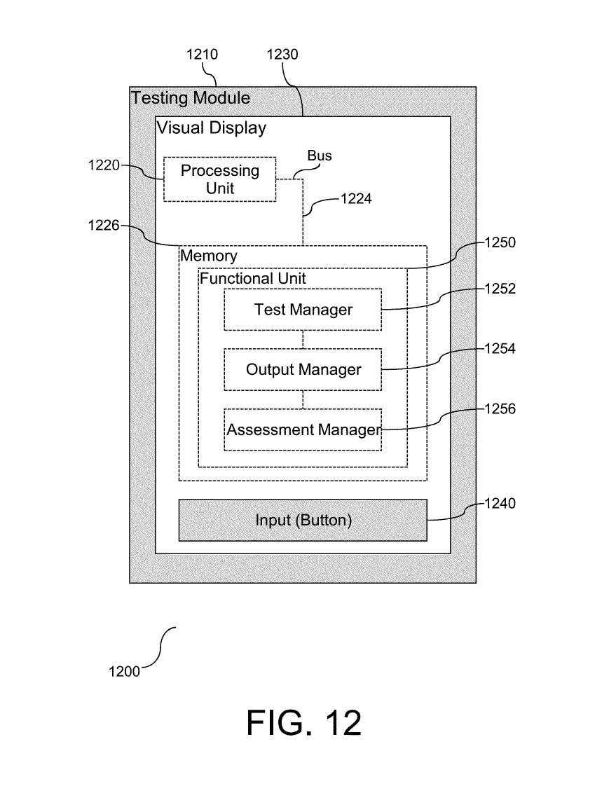

Cognitive and/or psychological testing may take place between a participant and a module, with the module having the functionality to support administration of the testing together with data acquisition and evaluation. FIG. 12 is a block diagram (1200) illustrating tools embedded in a testing module to support administration of Cognitive and/or psychological assessment. For illustrative purposes, a testing module (1210) is provided with a processing unit (1220) in communication with memory (1226) across a bus (1224). The testing module is provided with a visual display (1230) and an input element (1240) to communicate instructions to the processing unit (1220). The input element (1240) may be in the form of a button window on the visual display configured to receive input data, and various other forms of communication with the processor. In one embodiment, the input element may be a stylus to communicate with data on the visual display (1230).

A functional unit (1250) is provided in communication with memory (1226); the functional unit (1250) support neuro-cognitive and/or behavioral assessment. As shown, the functional unit (1250) is provided with a test manager (1252), an output manager (1254), and an assessment manager (1256). The test manager functions to administer a test battery, with the test battery including neuro-cognitive and/or behavioral test batteries. The output manager (1254), which is in communication with the test manager (1252), functions to receive output data pertaining to a compilation of reaction time data of the neuro-cognitive test presented on the visual display (1230). The assessment manager (1256), which is in communication with the output manager (1254), functions to analyze the output data as received from the output manager (1254) and to evaluate the basis for cognitive impairment. In addition, the assessment manager (1256) compares output data. The assessment manager (1256) may include, but is not limited to, current output data to one or more prior output data, or current output data to a sample population. In one embodiment, the output data from each of the tests is independently accessible from the memory (1226). Output data from the tests are presented in some form of a display, including a visual display, an auditory display, or a haptic display. In one embodiment, the assessment manager (1256) evaluates a behavioral profile associated with the behavioral test batteries to yield a score. Accordingly, the test manager (1252), output manager (1254), and assessment manager (1256) function to support administration and evaluation of neuro-cognitive and behavioral testing.

As identified above, the test manager (1252), output manager (1254), and assessment manager (1256), hereinafter referred to as tools, function as elements to support administration and evaluation of neuro-cognitive and behavioral testing, The tools (1252), (1254), and (1256), are shown residing in memory (1226) local to the testing module (1210). However, the tools (1252), (1254), and (1256) may reside as hardware tools external to memory (1226), or they may be implemented as a combination of hardware and software. Similarly, in one embodiment, the tools (1252), (1254), and (1256) may be combined into a single functional item that incorporates the functionality of the separate items. Accordingly, the managers may be implemented as software tools, hardware tools, or a combination of software and hardware tools.

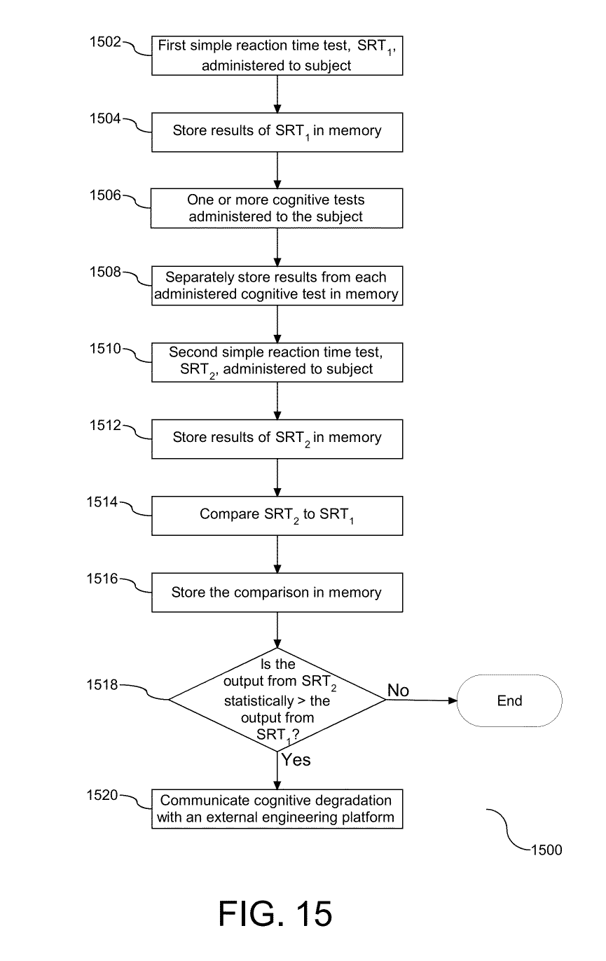

Referring to FIG. 15, a flow chart (1500) is depicted to illustrate one aspect of assessing cognitive efficiency. As shown, a first simple reaction time test, SRT₁, is administered to a subject (1502), and the results of the test are stored in memory (1504). Following the conclusion of SRT₁, one or more cognitive tests are administered to the subject (1506). Results from each administered cognitive test are separately stored in memory (1508). In one embodiment, the one or more cognitive tests are administered immediately after administration of SRT₁. Similarly, in one embodiment, the administration of cognitive tests is limited to a single test, or in one embodiment may include between two and five cognitive tests. Following the conclusion of the final cognitive test, a second simple reaction time test, SRT₂, is administered to the subject (1510), and the results of the SRT₂ are stored in memory (1512). Thereafter, a comparison of the first and second simple reaction time tests is conducted (1514), e.g. (SRT₁-SRT₂) or (SRT₂-SRT₁). The comparison of the tests is shown as being stored in memory (1516). In one embodiment, the results may be evaluated prior to storage, or may be communicated to a secondary location for evaluation and/or storage.

Comparison of the first and second simple reaction time tests based on the sequential order in which the tests are administered produces a unique data signature when compiling the result data. In one embodiment, the data received from the comparison of the first and second simple reaction time tests yields a significant brain vital sign of cognitive efficiency. The sequential administration of the tests as shown and described in FIG. 15 together with the tests used produces the unique data signature. In one embodiment, the signature is directly related to the integrity, order, and quantity of tests administered in the sequence shown and described in FIG. 15.

Comparison of the first and second simple reaction time test data is a comparison of data for a specific subject, e.g. patient. More specifically, the patient's second simple reaction time test data is compared to their test data for the first simple reaction time test. This measurement and subsequent comparison is employed to determine if there is a statistical difference in the test results, and if the comparison data shows a statistical value of a worsening cognitive condition, then it warrants a concern of an atypical data output. In one embodiment, following step (1516), it is determined if the comparison of the first and second reaction time test data shows a decrease, also referred to as a degrading cognitive condition (1516). A positive response to the determination at step (1516) is followed by communicating the cognitive degradation with an external engineering platform (1518) or in one embodiment, communicating the cognitive degradation to a healthcare professional. In one embodiment, the external engineering platform may include a software or hardware patient platform. A negative response to the determination at step (1516) concludes the evaluation of the administered simple reaction time tests.

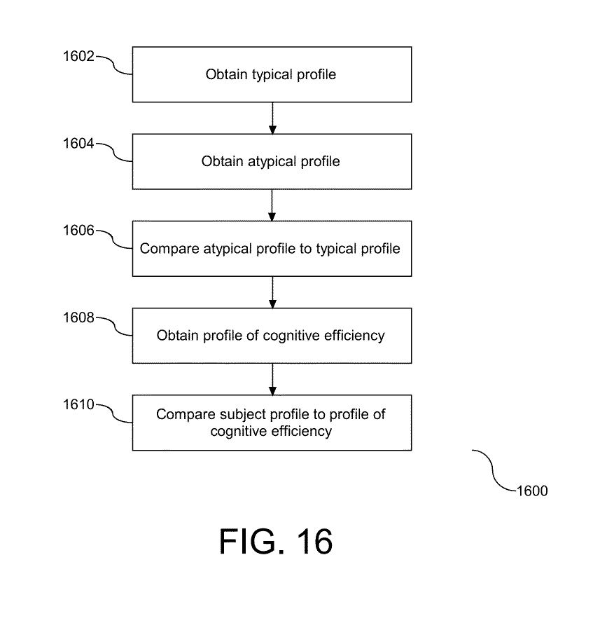

Referring to FIG. 16, a flow chart (1600) is provided illustrating comparison of the simple reaction times based on the sequential administration of tests shown in FIG. 15. Use of the sequential order and processing of the tests yields results that are referred to herein as a unique signature. Prior or subsequent to the administration of the sequential ordering of the tests, a normal or typical profile is obtained (1602). In one embodiment, the sequential ordering of the tests is conducted on a single subject that is typical and the comparison of the simple reaction time tests for this subject are identified and stored as a normal or typical profile or a normal or typical unique signature. Similarly, in one embodiment, the sequential ordering of the tests is conducted on two or more subjects that are normal or typical and the comparison of the simple reaction time tests for each subject is identified and stored as a normal or typical profile or a normal or typical unique signature. In one embodiment, statistical analysis is performed on the plurality of tests to create a mean and/or average signature for the normal or typical subject. Following step (1602), an atypical profile is obtained (1604). The process of obtaining the atypical profile is similar to the normal or typical profile except that the subject(s) for whom the data is gathered is in an atypical state. In one embodiment, there may be different levels of atypical states, and as such, more than one unique signature for an atypical state may be gathered and identified. Accordingly, at leave two unique signatures are obtained, including an atypical signature and a typical signature.

As shown in FIG. 15, a comparison of the first and second simple reaction time tests is conducted to obtain a vital sign of cognitive efficiency. Similarly, the atypical profile may be compared to the non-atypical profile (1606) to obtain a profile of cognitive efficiency (1608). In one embodiment, a range of values may be obtained from the profile data, including a range of values for cognitive efficiency, with the range indicating profiles that have a greater cognitive efficiency and a diminished cognitive efficiency. The cognitive efficiency from the tests being administered to the subject is compared to the profile of cognitive efficiency (1610). Results from the comparison of the subject to the profile are indicative of placement of the subject's cognitive efficiency with the range of profile cognitive efficiencies. In one embodiment, the cognitive efficiency results indicate whether the subject is in an atypical state. Accordingly, in addition to or separate from the signature, the profile comparison for cognitive efficiency is a tool employed to assess a typical or atypical state of the subject.

The unique signature obtained from the sequential test administration shown and described in FIG. 15 is due to the nature of the tests used, including the integrity, order, and quantity of the tests. In one embodiment, the comparison shown in FIG. 16 is based on comparison of the signature with a typical profile and comparison of the signature with an atypical profile. Similarly, in one embodiment, the unique signature functions similar to a thermometer, however in place of temperature measurement, the unique signature measures a state of the subject. Comparison of the measured state to a stored profile or set of profiles provides a measurement of a level of an atypical state, similar to the temperature measurement on a thermometer to a normal body temperature and a raised body temperature. Accordingly, the unique signature is obtained from the sequential delivery of the simple reaction time test with one or more cognitive tests there between, and functions as a unique tool for assessing an atypical state of the subject.

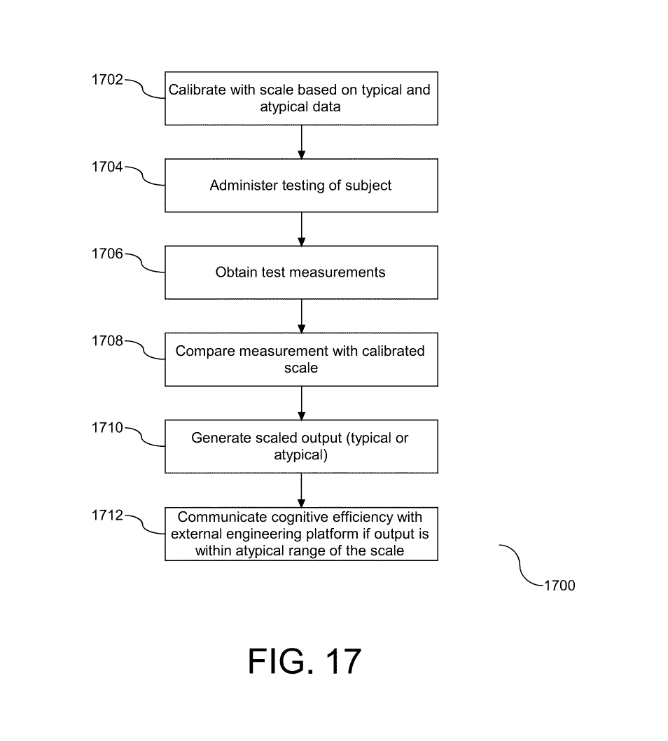

Referring to FIG. 17, a flow chart (1700) is provided illustrating a cognitive metering device employed with assessment data. The metering device functions similar to functionality of a thermometer with respect to a measurement scale, but is employed for cognitive data assessment. As shown, before the device can be used for evaluating assessment data, a scale is established for the device (1702). In one embodiment, the scale and calibration is based upon a set of typical and atypical data, including an associated data range. Once the scale and calibration is established, testing may be administered (1704), and output from the tests in the form of measurement data are obtained (1706). The measurements may be any cognitive data. In one embodiment, the measurement(s) may be a single measurement that is compared to the norm. More specifically, the measurement(s) from the assessment(s) is compared with the calibrated scale of the metering device (1708), and a scaled output is generated (1710). The scaled output indicates with the measurement(s) show that the data is in the typical range or the atypical range of the calibrated scale. For data that falls in the atypical range, this is indicative of a possible cognitive impairment. In one embodiment, the metering device communicates the cognitive impairment with an external platform (1712), such as a patient platform. Accordingly, the metering device is calibrated with data that represents typical and atypical measurements, so that assessment data can be measured with the metering device to determine cognitive impairment.

The cognitive metering device shown and described in FIG. 17 is calibrated and scaled with a set of data. It is understood that cognitive assessment data may be subject to change, and furthermore, in different environments data may have different interpretation. Furthermore, the scale in the metering device may be different based upon a different data set having a different data range. Accordingly, there are various factors that may require a re-calibration or re-scaling of the device.

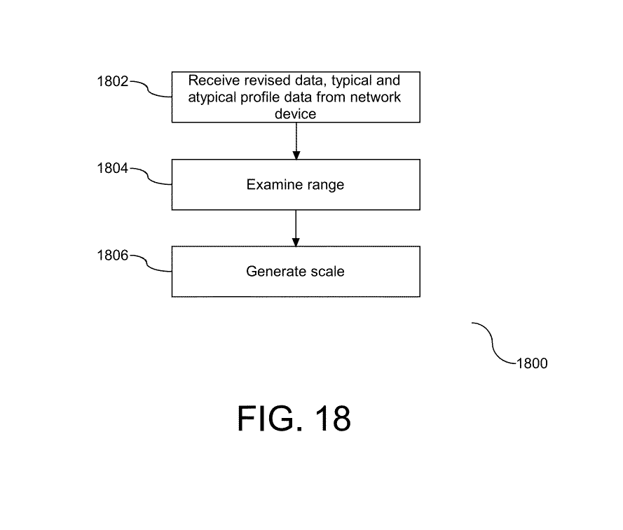

Referring to FIG. 18, a flow chart (1800) is provided illustrating a process for calibrating or re-calibrating the metering device. The metering device receives a revised cognitive data set (1802), with the revised data including values representing a typical profile and an atypical profile. In one embodiment, the metering device receives the revised data set from a network device. The range associated with the revised data set is examined, together with the profile representing a typical profile and an atypical profile (1804). Thereafter, a scale for the received data is generated (1806). Accordingly, the metering device may be re-calibrated in response to receipt of revised cognitive data.

The cognitive assessment device described herein may be configured with test batteries that are preconfigured for specific assessments. In one embodiment, the assessment device may operate in a dynamic. More specifically, the assessment device may be configured with hardware for administering the assessment(s).

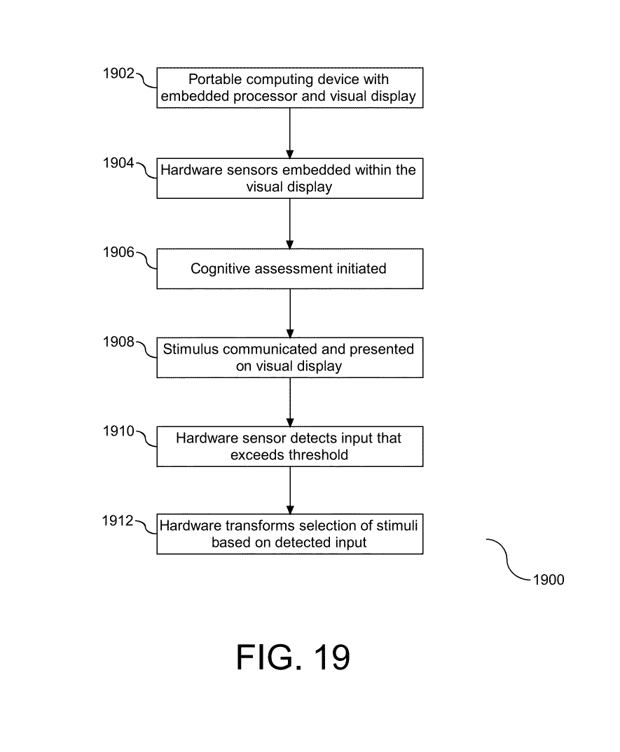

Referring to FIG. 19, a flow chart (1900) is provided illustrating a process for employing the cognitive assessment device, as described above, with one or more embedded hardware sensors. A portable computing device is configured with an embedded processor and associated visual display (1902). One or more hardware sensors are embedded with or in communication with the visual display (1904) Examples of the embedded hardware sensors include, but are not limited to, a resistive sensor, a capacitive sensor, or combinations thereof. A resistive sensor is a transducer or electromechanical device that converts a mechanical charge, such as displacement, into an electrical signal that can be monitored. Similarly, a capacitive sensor is measures the force associated with or received by the sensor in communication or embedded with the visual display. At such time as the cognitive assessment tool is initiated (1906) via the embedded processor, stimulus is communicated and presented on the visual display (1908), and the presentation of the stimulus warrants a response. More specifically, the response is received by a physical interaction with the visual display. One or more of the embedded hardware sensors of the visual display detect input (1910). In one embodiment, the sensors are configured with a threshold, and the detected input would have to meet or exceed the threshold to be considered as stimulus response data. The device hardware transforms selection and presentation of a second or subsequent stimuli based on the detected input (1912). The visual display embedded sensor(s) function as a hardware device to receive input responsive to presentation of stimuli. In one embodiment, the visual display sensor(s) also function to communicate with the processing unit for test battery selection or subsequent stimuli selection or presentation. Accordingly, the embedded hardware sensor(s) of the visual display transforms selection and presentation of stimuli and associated cognitive and/or behavioral assessment(s).

As described above, the cognitive assessment device is portable and employed in various environments responsive to different activating factors. It is understood and recognized that the assessment device may be placed in a low power state when the device is not active, e.g. not in the process of conducting an assessment. In one embodiment, the assessment device may be transformed to transition from the low power state to an active state based on an external device.

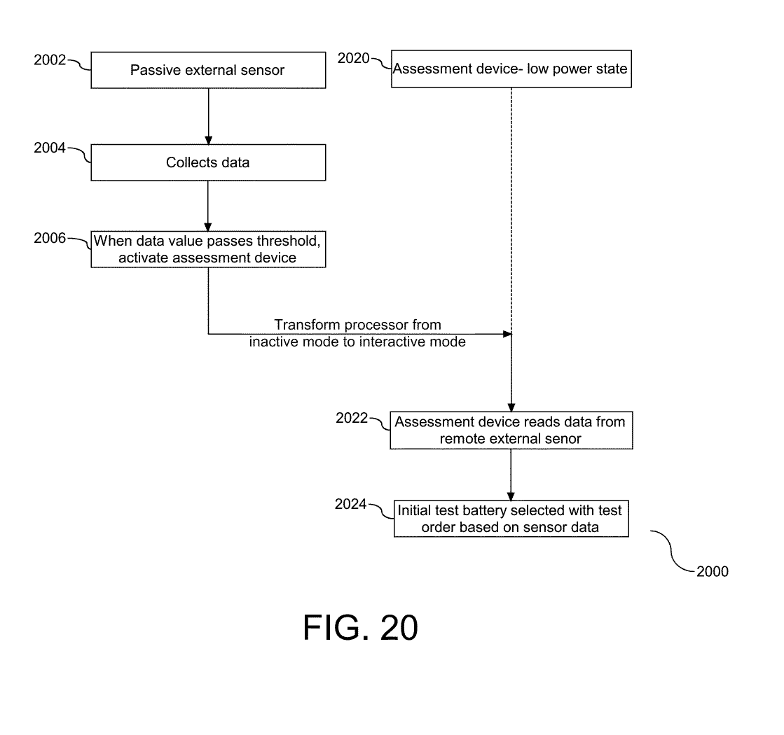

Referring to FIG. 20, a flow chart (2000) is provided illustrating a process for interacting with the portable assessment device, with the interaction affecting the state of operation of the device. As shown, a passive external device is provided physically detached from the portable assessment device (2002). The passive device functions to collect data (2004), and in one embodiment sends the collected data to the portable assessment device (2006). In one embodiment, the passive device communicates with the portable assessment device through an open application program interface. While the passive device is collecting data, the portable assessment device operates in a low power state (2020), examples including but not limited to sleep mode, standby mode, hibernate mode, or in one embodiment an alternate low power mode. The sleep mode and standby mode are low power states where the visual display and any persistent storage devices are turned off, but the memory chip, such as RAM, is continuously refreshed. In addition, the processing unit is throttled down to a low power state. In one embodiment, an alternate power saving mode, such as a hibernate mode, may be utilized by the assessment device.

When the data collected by the passive external sensor attains a value that exceeds a threshold, the portable cognitive assessment device is activated (2006). More specifically, the operating state of the portable assessment device is transformed from the low power state to an active mode. In one embodiment, the passive external sensor, and more specifically, the data from this sensor, controls activation of the assessment device. In one embodiment, the passive external sensor communicates with the assessment device through a wireless communication protocol, such as Bluetooth. The passive external sensor may include, but is not limited to, a helmet sensor, a sensor attached to a bracelet, and other forms of passive sensors. Following the activation, the assessment device, reads the data received from the remote external sensor (2022). An initial test battery is selected based on the received sensor data (2024). In one embodiment, the sensor data controls the test selection. In another embodiment, a profile of a signal received by the assessment device from the passive sensor will dictate the test selection. As described above, test data is received and analyzed. In one embodiment, real-time results of the data received from the test battery can be determinative of selection of one or more additional assessments. The combination of the passive sensor in communication with the assessment device enables the assessment device to operate in a low power state until such time as the data collected form the sensor warrants an assessment. Accordingly, the passive sensor functions as an external hardware tool that transforms the operating state of the assessment device, and more specifically, transforms the state from a low power state to an interactive mode for assessment.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described above with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowcharts and block diagrams in the Figures illustrate the architecture, functionality, and operation of possible implementations of systems, methods and computer program products according to various embodiments of the present invention. In this regard, each block in the flowcharts or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated. Accordingly, the enhanced assessment module supports cognitive and behavioral assessment of a participant subject in the field, and at the same time provides a unique employment of test and associated test batteries for the assessment.

### ALTERNATIVE EMBODIMENT

It will be appreciated that, although specific embodiments of the invention have been described herein for purposes of illustration, various modifications may be made without departing from the spirit and scope of the invention. Accordingly, the scope of protection of this invention is limited only by the following claims and their equivalents.

What is claimed is:

1. An apparatus comprising:
a processor in communication with memory and a visual display;
a testing module in communication with the memory;
the testing module comprising a plurality of test batteries, including at least a first test battery and a second test battery, the first test battery comprising:
a first simple reaction time test;
at least one cognitive test; and
a second simple reaction time test;
a sensor in communication with the testing module, data measured by the sensor to be communicated to the processor;
output from the first test battery activating the second test battery, the second test battery comprising at least one test different from the first test battery; and
output data from the first and second simple reaction time tests and from the cognitive test stored in memory, and the output from each of the tests being independently accessible from the memory.

2. The apparatus of claim 1, further comprising an evaluation for cognitive impairment, the evaluation including comparison of a second output from the second simple reaction time test with a first output from the first simple reaction time test.

3. The apparatus of claim 2, wherein the second output having a value statistically greater than the first output indicates cognitive degradation.

4. The apparatus of claim 2, further comprising a cognitive efficiency indication from the first measurement, wherein the cognitive efficiency includes speed and accuracy.

5. The apparatus of claim 1, further comprising a sequential administration of the tests, including administration of the at least one cognitive test following conclusion of the first simple reaction time test.

6. The apparatus of claim 4, further comprising administration of the second simple reaction time test following conclusion of the at least one cognitive test.

7. The apparatus of claim 2, further comprising an evaluation for cognitive impairment, the evaluation including comparison of the first measurement with a stored profile.

8. The apparatus of claim 6, further comprising an evaluation for cognitive impairment, the evaluation including comparison of the first output with second output.

9. The apparatus of claim 1, further comprising the processor to operate in a low power state during a non-active mode of operation.

10. The apparatus of claim 9, further comprising a passive external sensor in communication with the processor, the sensor physically displaced from the processor.

11. The apparatus of claim 10, further comprising the external sensor to acquire external data and to transform the operating state of the processor based on a value associated with the acquired data, including the sensor to generate a signal to transition the processor from an inactive state to an active state.

12. The apparatus of claim 11, further comprising selection of an initial test battery based on a profile of the signal.

13. The apparatus of claim 1, further comprising a cognitive metering device in communication with the testing module, the cognitive metering device to receive the output data and to compare the output data with a calibrated scale and to generate an output.

14. The apparatus of claim 13, wherein the cognitive metering device includes a typical range and an atypical range, with data within the atypical range indicative of cognitive impairment, and further comprising an output signal created and communicated to an external patient platform for data measured within the atypical range.
